(19) United States
(12) Patent Application Publication
(10) Pub. No.: US 2014/0163518 A1
(43) Pub. Date: Jun. 12, 2014

(54) Safety Syringe

(71) Applicant: Jack Epstein, Punta Gorda, FL (US)

(72) Inventor: Jack Epstein, Punta Gorda, FL (US)

(21) Appl. No.: 13/711,771

(22) Filed: Dec. 12, 2012

(51) Int. Cl. A61M 5/32

(52) U.S. Cl. CPC A61M 5/3232
USPC 604/506; 604/110

(57) ABSTRACT

A safety syringe which includes an outer casing having a closed and an open end, a transition chamber having a closed and an open end, a spring assembly, and a plunger. The transition chamber is held for sliding movement within the outer casing and defines a needle concealment chamber, while the plunger defines a needle receiving chamber and is held for sliding movement within the transition chamber. A liquid medicine chamber within the transition chamber holds a precharged quantity of injectable medicine. The spring assembly is moved from its initial compressed configuration, including a needle within a needle containment chamber, when movement of the plunger and the transition chamber are moved together within the outer casing to extend the needle from a needle exit hole in the end of the outer casing. The medicine is ejected from the needle upon further movement of the plunger within the transition chamber, after which further movement of the plunger within the transition chamber retracts the needle to eject safely into the needle receiving chamber.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to medical devices and more specifically to a preloaded tamper-proof retractable syringe suitable for mass production which has a low trigger force that post injection renders itself non-reusable. The injection cycle starts with the needle in a retracted or concealed needle position and ends with the cannula in a fully retracted position.

2. Description of Related Art

A major cause of the spread of infectious disease in the general population is due to the presence of exposed hypodermic syringes. Infection can be spread from accidental needle sticks by way of exposed, contaminated needles used in high risk patients in hospitals and medical facilities. Used syringes with extended needles present a risk to medical personnel, sanitation employees and others in the disposal chain.

The prior art has not recognized the potential for spread of infectious disease related to the accidental exposure of uncapped needles with regard to the general population when there is the presence of exposed, contaminated hypodermic syringes. Infection can be spread from patients in hospitals and medical facilities through accidental needle sticks from syringes used on high risk patients. No medical personnel, sanitation employees or other category of employee involved in the disposal of contaminated medical waste is protected from the great risk that is present from the potential threat of accidental needle stick by used syringes with extended needles.

All prior retractable hypodermic syringe designs have exposed needles that will retract at the end of the injection cycle. However, this prior art has not produced a non-reusable tamper-proof hypodermic syringe that has the needle in an internal concealed and retractable position.

This disclosure is generally directed to a tamper-proof hypodermic syringe that is simple, reliable, cost effective, easy to use, manufacture and assemble which can be also be categorized as a “non-exposed” type.

The foregoing examples of the related art and limitations related therewith are intended to be illustrative and not exclusive. Other limitations of the related art will become apparent to those skilled in the art upon a reading of the specification and a study of the drawings.

### BRIEF SUMMARY OF THE INVENTION

This disclosure is directed preferably to an automatically extendable and retractable, tamper proof, self-destructing, single-use pre-filled safety syringe. The cannula or needle is initially unexposed or concealed prior to use and extends in a single forward motion, injects fluids, and then retracts back into a needle receiving chamber. The needle is extended outwardly only when medication is administered and, post injection, the needle automatically retracts back into the needle receiving chamber for disposal after a one-time usage.

The safety syringe which includes an outer casing having a closed and an open end, a transition chamber having a closed and an open end, a spring assembly, and a plunger. The transition chamber is held for sliding movement within the outer casing and defines a needle concealment chamber, while the plunger defines a needle receiving chamber and is held for sliding movement within the transition chamber. A liquid medicine chamber within the transition chamber holds a precharged quantity of injectable medicine. The spring assembly is moved from its initial compressed configuration, including a needle within a needle containment chamber, when movement of the plunger and the transition chamber are moved together within the outer casing to automatically extend the needle from a needle exit hole in the end of the outer casing. The medicine is ejected from the needle upon further movement of the plunger into the transition chamber, after which, further movement of the plunger within the transition chamber retracts the needle to fall safely into the needle receiving chamber. After the liquid medicine is injected, a preloaded compressed spring is released to force the needle cannula to retract and to spring out of the transition chamber into the needle receiving chamber thus concealing the needle and providing a fully tamper-proof syringe.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference to the drawings and by study of the following descriptions.

### BRIEF DESCRIPTION OF THE SEVERAL VIEWS OF THE DRAWING(S)

FIGS. 1 and 1A are longitudinal section views of the safety syringe 10 in an initial position ready for use.

FIGS. 2 and 2A are views of FIGS. 1 and 1A with safety cap 20 removed.

FIGS. 3 and 3A are views of FIGS. 2 and 2A showing the plunger 18 at a first stage of movement with the cannula 54 extending into the subject and commencing fluid injection.

FIGS. 4 and 4A are views of FIGS. 3 and 3A showing the plunger 18 at a second stage of movement, completing injection of the fluid into the subject.

FIG. 4B is an enlarged view of area 4B in FIG. 4.

FIGS. 5, 5A and 5B are views of FIGS. 4 and 4A showing the plunger 18 at a third stage of movement, the needle base holder 58 applying force to dislodge distal plug 48.

FIGS. 6 and 6A are views of FIGS. 5 and 5B showing the plunger 18 at a fourth stage of movement, the spring assembly 16 being released after forced displacement of the distal plug 48.

FIG. 7A is a further step of the fourth stage shown in FIGS. 6 and 6A, the needle 54 and needle base holder 58 automatically falling free into the needle receiving chamber 40.

FIG. 7B is a view of FIG. 7A showing the plunger 18 at a fifth stage of movement, the entire spring assembly 16 falling free into the receiving chamber 40, and the distal ring stop 34 locking the plunger 18 from further movement, the cap 20 also being replaced.

FIG. 8 is an enlarged view of one end of FIG. 6A.

FIG. 9 is a section view of the plunger body 30.

FIG. 10 is a section view of the needle base holder 58.

FIG. 11 is a section view of the distal stop ring 34.

FIG. 12 is a section view of the proximal ring plug 38.

FIG. 13 is a section view of the spring holder 36.

FIG. 13A is a section view of the distal plug 48.

FIG. 14 is a section view of the syringe body 24.

FIG. 15 is an end elevation view of the syringe body 24 of FIG. 14.

FIG. 16 is a section view of the transition body 28.

FIG. 17 is a section view of the spring assembly 16.

FIG. 18 is an enlarged view of one end portion of FIG. 1.

FIG. 19 is an enlarged view of area 19 in FIG. 18.

Exemplary embodiments are illustrated in reference figures of the drawings. It is intended that the embodiments and figures disclosed herein are to be considered to be illustrative rather than limiting.

### DETAILED DESCRIPTION OF THE INVENTION

### Nomenclature

10. safety syringe
12. outer casing
14. transition chamber
16. spring assembly
18. plunger
19. medicine chamber
20. cap
21. spring sleeve chamber
22. needle concealment chamber
24. syringe body
26. finger tabs
28. transition chamber body
30. plunger body
32. thumb depressor
34. distal stop ring
36. transition chamber end
38. proximal ring plug
39. friction interface
40. needle receiving chamber
41. friction interface
42. annular groove
44. stop
45. ring plug landing
46. ring stop
47. spring sleeve chamber relief
48. plug
50. proximal contact surface
52. cap retaining teeth
54. needle
56. needle exit hole
58. needle base holder
60. medication entrance cone
62. distal plug seat
64. spring
66. plunger sealing surface
68. spring sleeve
70. spring center shaft
72. needle guide hole
74. spring seat
76. spring assembly support hole
78. plunger body stop
80. needle support hole
82. center shaft seat
84. spring seat cavity
86. medicine chamber
88. spring seat
90. center shaft end

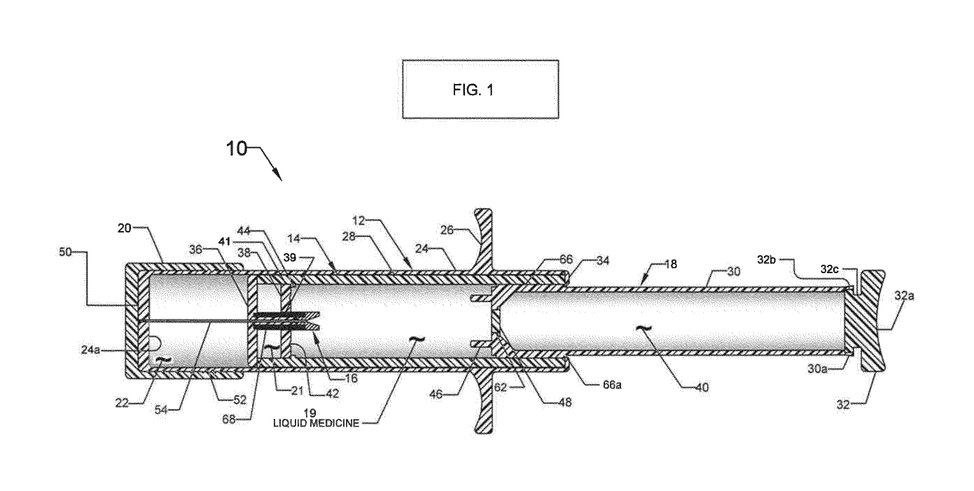

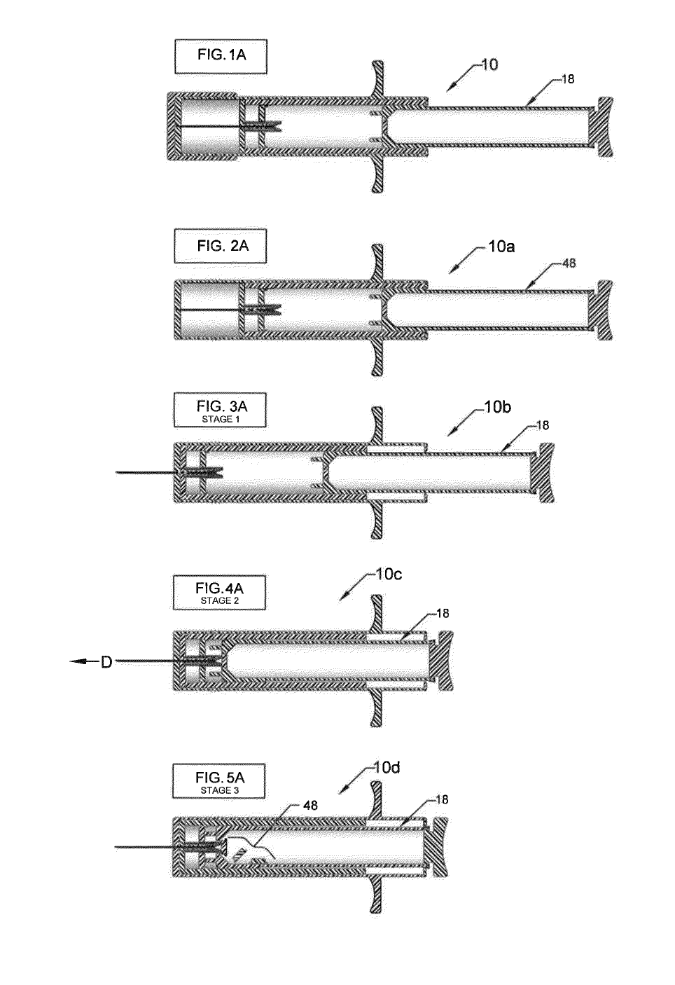

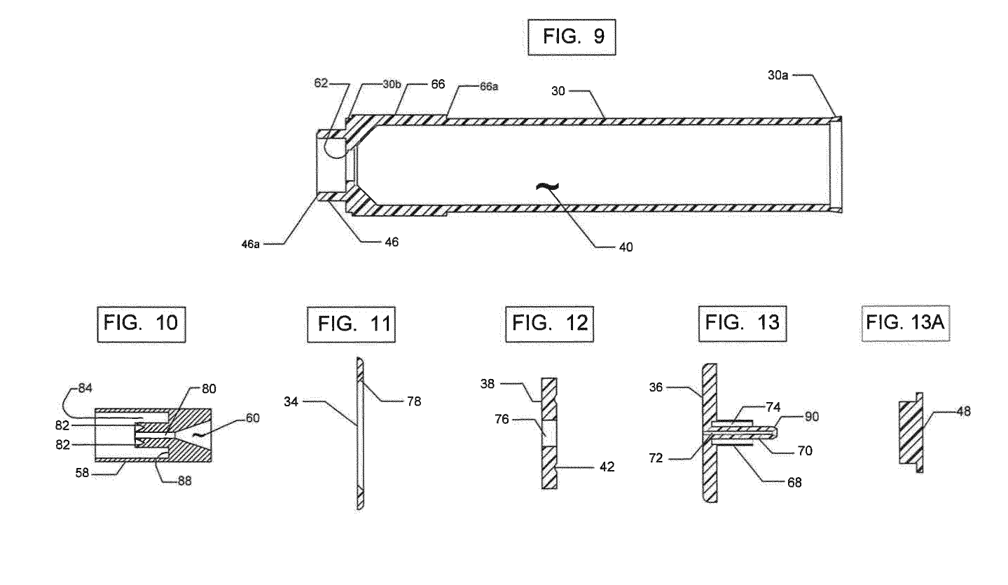

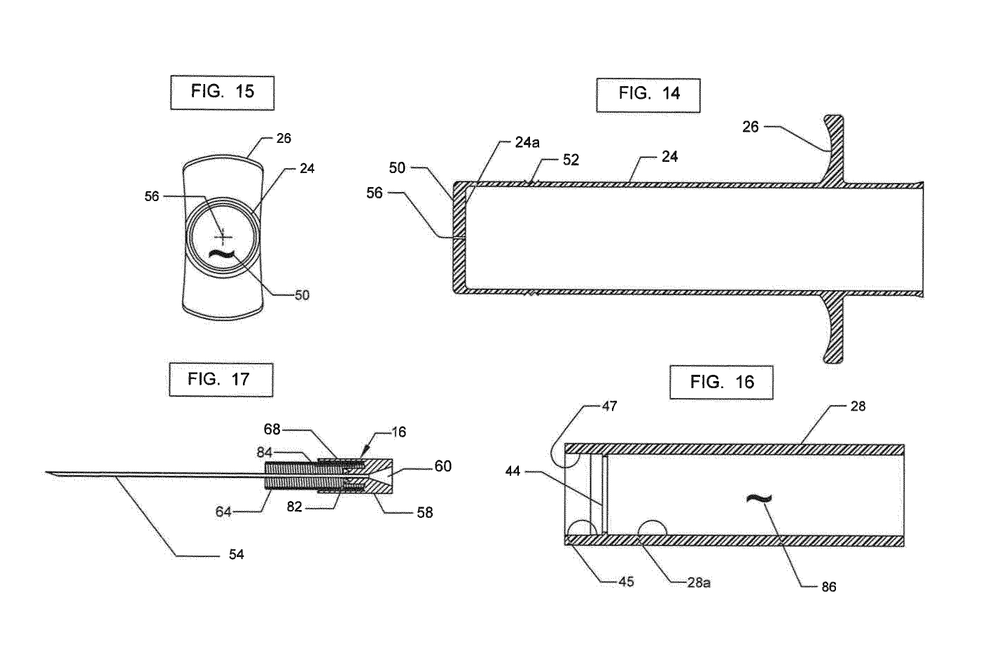

Referring now to the drawings, and firstly to FIGS. 1 and 9 to 16, the safety syringe, shown generally at numeral 10, includes an outer casing 12, a transition chamber 14, a spring assembly 16 and a plunger 18. The outer casing 12, mold formed of any suitable surgical plastic material or an equivalent, includes an elongated tubular syringe body 24 having a closed end 24a defining a proximal contact surface 50 having a central axially extending needle exit hole 56 formed therethrough. Outwardly extending from adjacent the open end of the syringe body are finger tabs 26.

The transition chamber 14 is formed of an elongated tubular open ended transition body 28 defining a medicine chamber 86 within the hollow interior thereof. The transition body 28 also includes an inwardly extending stop or shoulder 44 and a slightly reduced in diameter ring plug landing 45 with respect to the smooth cylindrical bore 28a of the transition body 28. Again, the transition body 28 is preferably formed of molded surgical plastic material or other material suitable for this purpose in a syringe. A slightly enlarged in diameter spring sleeve chamber relief 47 defines the wall interior surface of a spring sleeve chamber 21.

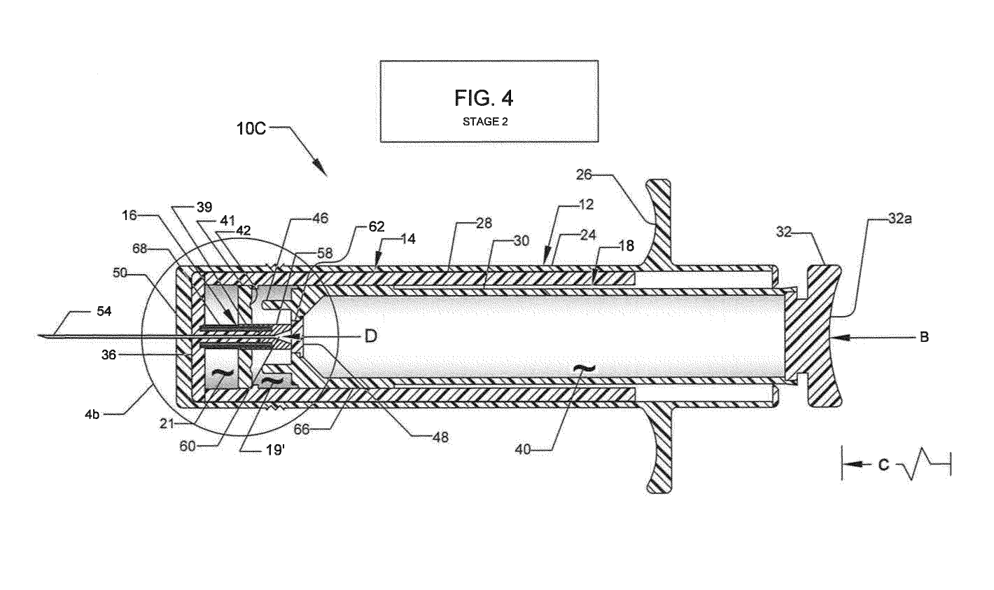

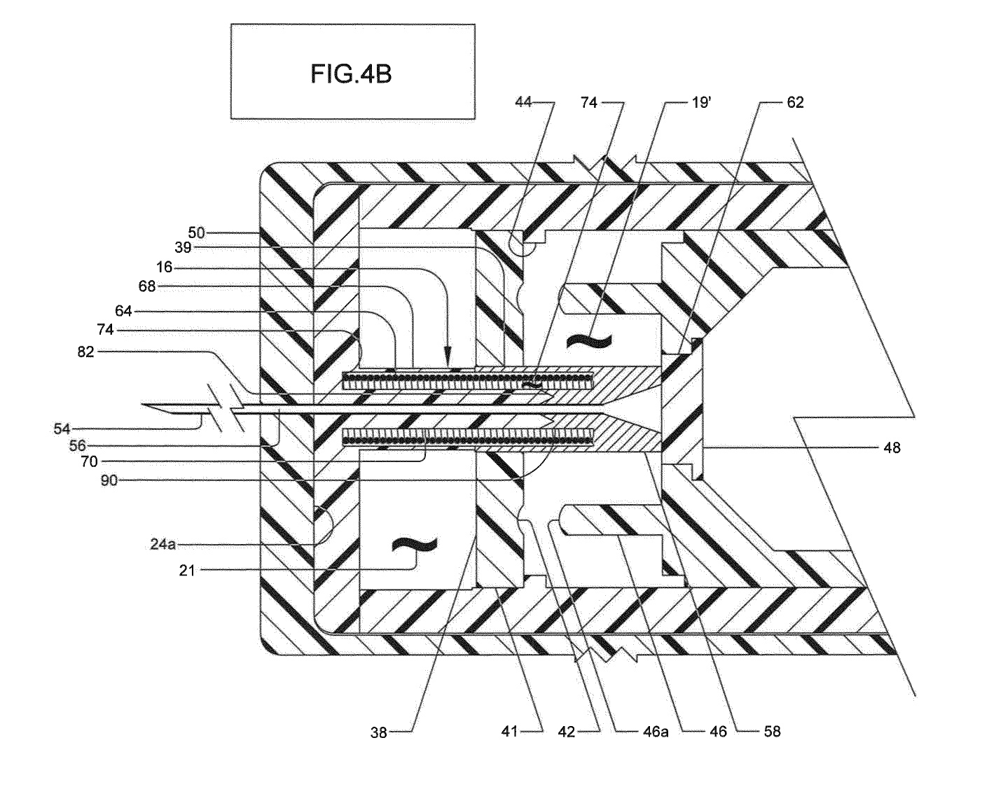

The spring assembly 16, as best seen in FIGS. 4B, 18 and 19, includes a machined metal needle base holder 58 having a spring seat cavity 84 sized to receive an elongated compression spring 64 which is seated within the spring seat cavity 84 and around a stabilizing spring seat 88. The needle base holder 58 further includes a conically shaped opening medicine entrance cone 60 which receives and funnels liquid medicine held within the syringe for dispensation from an elongated needle or cannula 54, the proximal end of which is tightly secured within a longitudinally extending needle support hole 80 formed centrally through the needle base holder 58. As seen in these referenced figures, the spring assembly 16 is shown with the spring 64 in a fully compressed initial configuration.

The plunger 18 includes an elongated cylindrical plunger body 30 and a thumb depressor 32, each preferably mold formed of surgical plastic or other suitable material. Formed preferably as a molded single unit, the plunger body 30 includes an enlarged outer plunger sealing surface 66 disposed adjacent the proximal end of the plunger body 30. An outwardly extending shoulder 66a defines the proximal end of the plunger sealing surface 66, while a groove 30a is formed and outwardly extends from the cylindrical inner surface of the inner cylindrical surface of the plunger body 30 which defines the needle receiving chamber 40. Defining the proximal end of the plunger body 30 is an annular-shaped ring stop 46 having a circular proximal edge 46a. Adjacent to this ring stop 46 is a distal plug seat 62 sized to tightly receive a distal plug 48 (FIG. 13A) forcibly installed therein.

The thumb depressor 32 having a concave thumb surface 32a and an annular inwardly extending groove 32c and an enlarged ring shaped area 32b is tightly frictionally assembled into the groove 30a of the distal end of plunger body 30. A plug 48 is tightly frictionally assembled into the distal plug seat 62. A flat distal stop ring 34 having an inwardly facing knife edge shaped plunger body stop 78 is secured onto the open end of the syringe body 24 and serves several purposes, the first of which upon assembly longitudinally aligns the proximal end 66a with the open end of the transition body 28. This stop ring 34 also prevents withdrawal of the plunger 18 from the transition chamber body 28 by making contact against shoulder 66a.

The safety syringe 10, in its preferred embodiment ready for use as shown in FIG. 1, includes a quantity of liquid medicine held within a medicine chamber 19 with the plunger 18 in its fully extended position. The spring assembly 16 as best seen in FIG. 4B is held with the spring 64 in its fully compressed configuration seated within a spring seat 74 formed by a spring sleeve 68 of the spring holder 36. The spring holder 36 is preferably formed of molded or machined surgical plastic material or other suitable material for this purpose. A central spring center shaft 70 closely fits within the inner cylindrical surface of the compressed spring 64 and includes a longitudinally extending needle guide hole 72 formed axially therethrough. The center shaft end 90 is v-shaped so as to matingly engage within a center shaft seat 82 formed into the distal edge of the spring seat 88.

To hold the spring 64 in its compressed initial configuration, as best seen in FIGS. 4B, 18 and 19, the outer cylindrical surface of the needle base holder 58 is tightly frictionally engaged into an axially positioned spring assembly support hole 76 formed longitudinally through the proximal ring plug 38. This tight frictional engagement at 39 is sufficient to retain the needle base holder 58 tightly within the spring assembly support hole 76, all compression forces acting between the spring holder 36 which is permanently attached to the end of the transition body 28 and the proximal ring plug 38 against the stop 44. Therefore, on assembly, the spring sleeve chamber 21 defined therebetween is established and maintained.

### Sequence of Operation

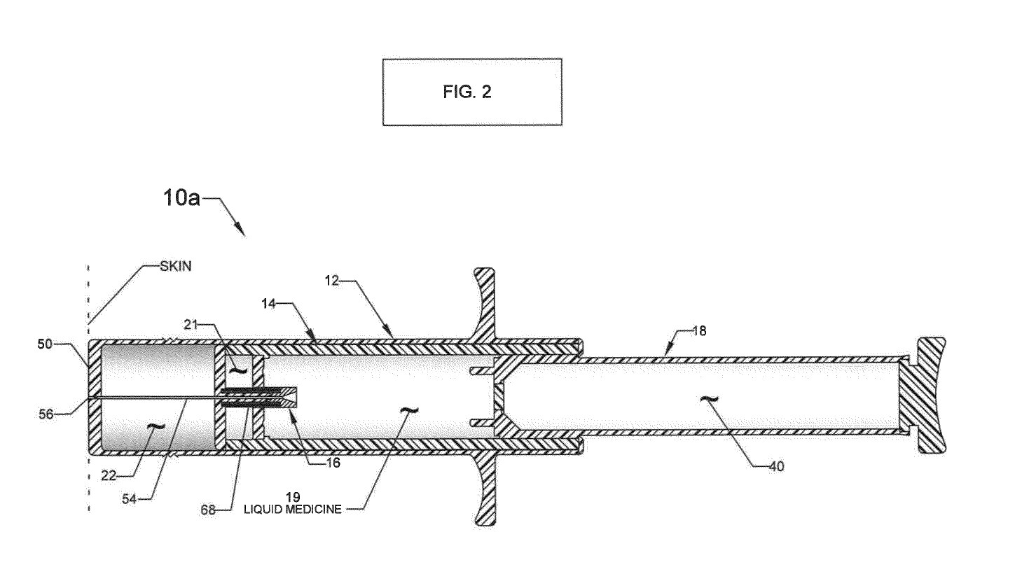

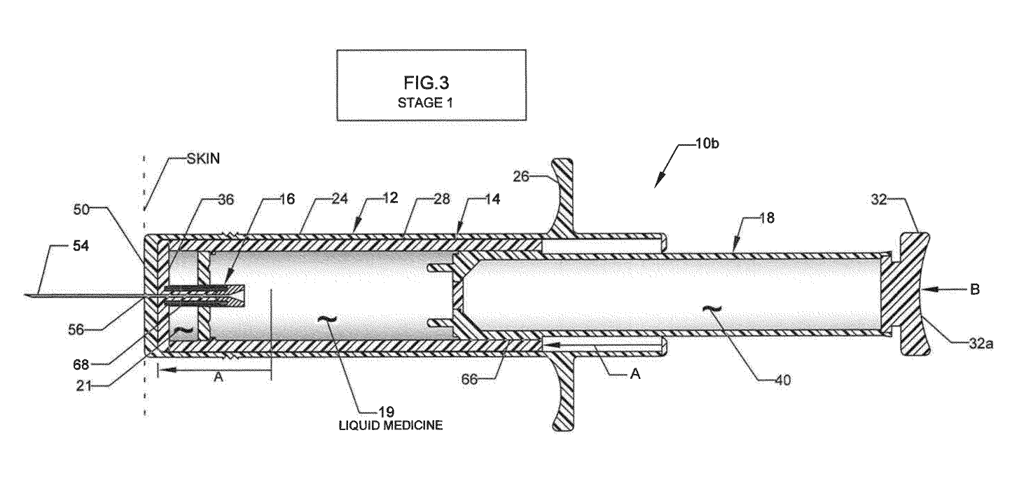

In use, after a protective cap 20 has been removed from the closed end of the safety syringe 20 at 10a as seen in FIGS. 2 and 2A, the proximal contact surface 50 is placed against the skin of a patient who will receive the to-be-injected liquid medicine within the medicine chamber 19. Stage 1 of injection sequence shown in FIGS. 3 and 3A occurs at 10b when the plunger 18 is moved inwardly into the outer casing 12 by applying thumb force in the direction of arrow B against surface 32a. Because the liquid medicine in the medicine chamber 19 is essentially non-compressible, and because air is within the needle containment chamber 22 defined between the closed end of the transition body 28, and a spring holder plate 36, air within this needle concealment chamber 22 is expelled outwardly through the clearance between the needle 54 and the needle exit hole 56 until the closed end of the syringe body 24 moves distance A and comes in contact with the spring holder plate 36. The needle 54 is now simultaneously extended and has penetrated into the skin of the patient.

Now referring to FIGS. 4, 4A and 4B, stage 2 of the sequence of use of this device 10 at 10 is there shown at numeral 10c wherein the plunger 18 is further forced into the outer casing 12 distance C by manually grasping the previously described finger tabs 26 and applying further pressure in the direction of arrow B against the thumb depressor 32. However, in stage 2, the closed end of the transition chamber 14 formed by the spring holder 36 comes in contact with the closed end of the syringe body 24 and completely exhausts all of the air within the needle concealment chamber 22. Note that the plunger 18 is here moving within the transition chamber 14 by tight sealed sliding engagement between the inner cylindrical surface of the transition body 28 and the plunger sealing surface 66. This second stage of movement of the plunger 18 causes substantially all of the liquid medicine within the liquid medicine chamber 19 to be injected in the direction of arrow D through needle 54 into the patient.

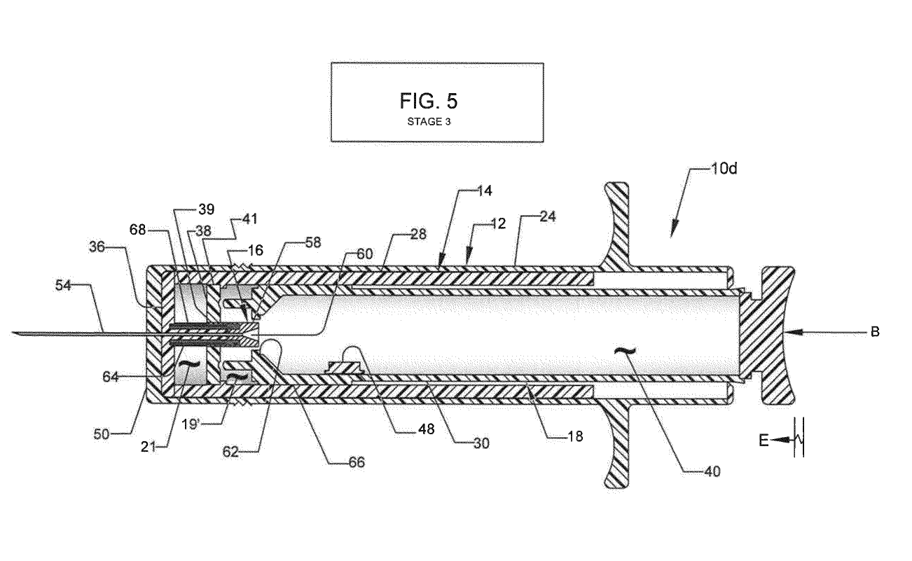

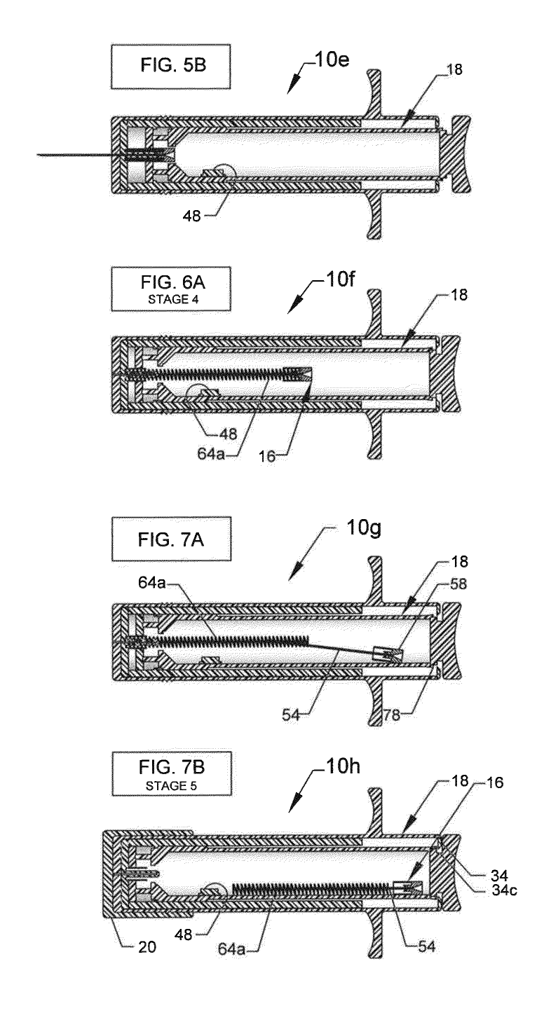

Referring now to FIGS. 5, 5A and 5B, stage 3 of deployment of the safety syringe 10 is there shown generally at numeral 10d. In stage 3, the plunger 18 is moved a further distance into the transition chamber 14 such that the free end of the needle base holder 58 is forced into contact with the distal plug 48 which, as previously described is held frictionally within the distal plug seat 62 and now becomes dislodged and falls into the needle receiving chamber 40. Note that the size of the spring sleeve chamber 21 and the reduced size of the medicine chamber 19′ is maintained in stage 3. Note also that the friction forces remain intact to hold the spring 64 in its fully compressed configuration.

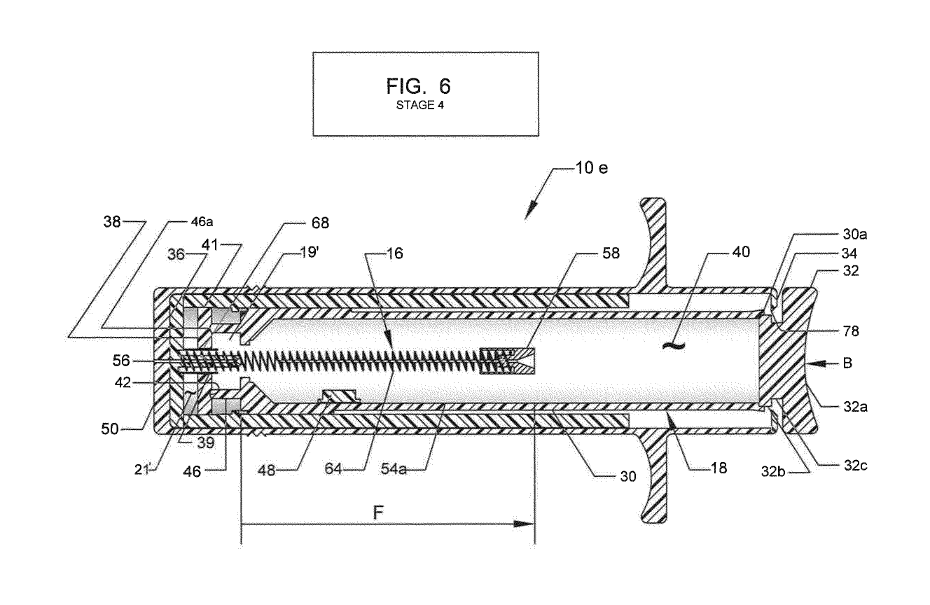

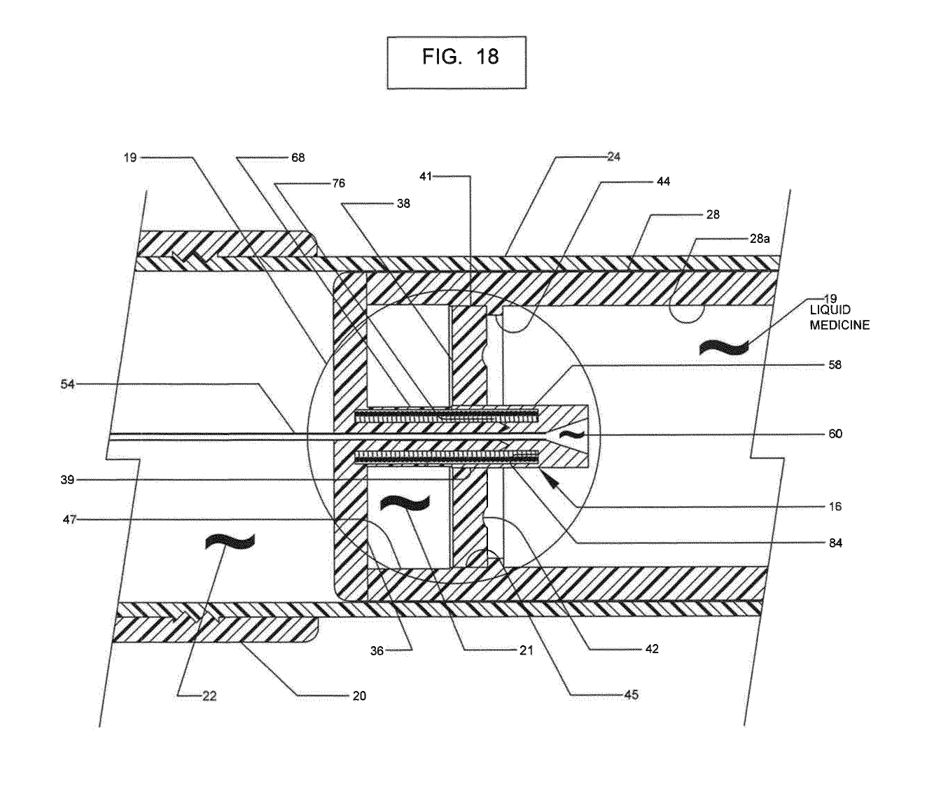

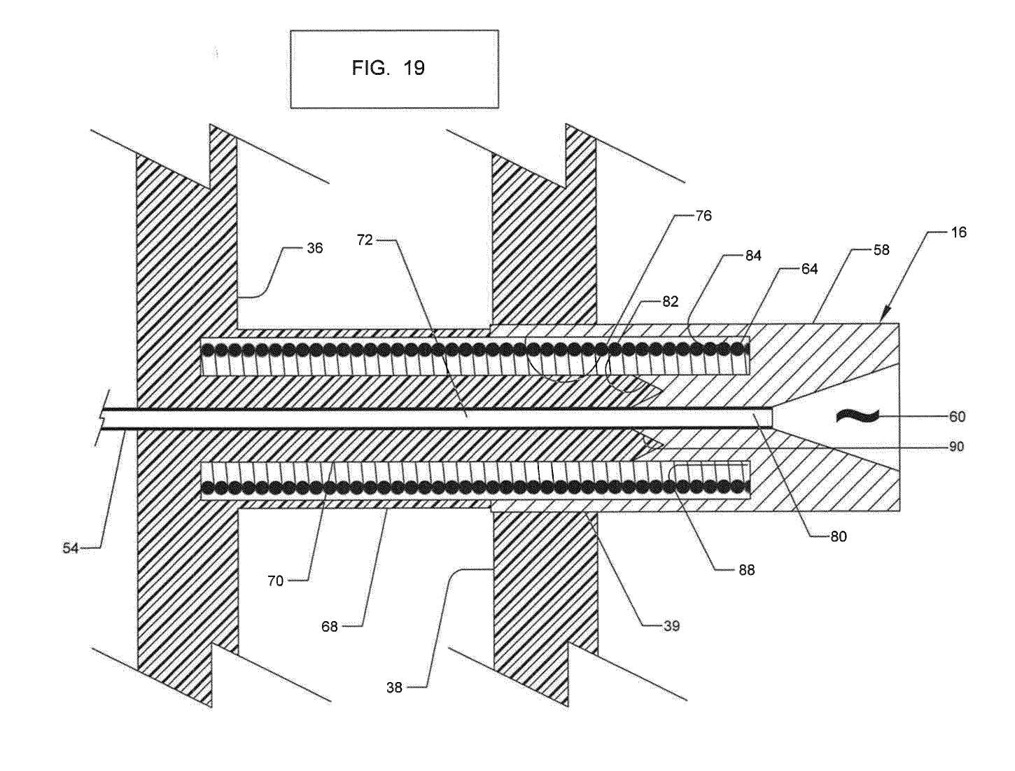

Referring now to FIGS. 6 and 6A, stage 4 of deployment of the safety syringe is there shown generally at numeral 10e. In stage 4, the plunger 18 has been forced by pressure against thumb depressor 32 into contact between the circular edge 46a of the ring stop 46 and the annular groove 42 in the proximal ring plug 38 forcing the plug 38 to move away from stop 44 and off ring plug landing 45. Referring also to FIGS. 18 and 19, this causes movement of the proximal ring plug 38 on the needle base holder 58 a distance sufficient to completely release the needle base holder 58 from friction engagement within the spring assembly support hole 76 and releasing the spring to quickly extend to its free length. Then, as seen in FIG. 7A, the needle 54 and needle base holder 58 will start to fall into the needle receiving chamber 40, the needle 54 being rendered retracted and completely concealed within the needle receiving chamber 40.

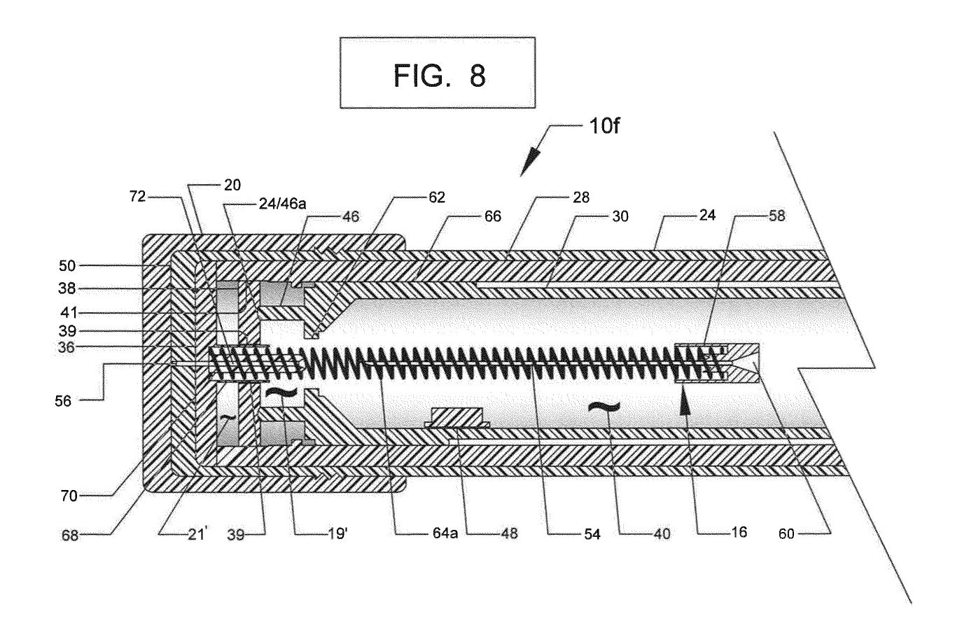

Referring now to FIGS. 7B and 8, stage 5 of deployment of the safety syringe is there shown generally at numeral 10f. In stage 5, the plunger 18 is shown fully inserted into the syringe body 24 whereupon the proximal ring plug 38, previously forced into contact with the spring holder 36, eliminates the reduced in size spring sleeve chamber 21′ altogether. This movement overcomes the friction interface 39 as well as continuing to overcome the friction interface 41. At the end of stage 5, the inner edge of the plunger body stop 78 biasingly engages into the locking groove 34c of the distal ring stop 34 so as to prevent any withdrawing movement of the plunger 18 thereafter. The entire spring assembly 16 including the needle 54 and plug 48 have now fallen safely into the needle receiving chamber 40 and, when the cap 20 is repositioned back onto the closed end of the syringe body 24, the entire assembly 10f is fully secured from any harm or puncture risk associated with its handling ready for disposal.

Note that it is preferred that the stages of deployment be seamless and smooth in operation so that the movement of the plunger through the various stages is stepless starting with the removal of the cap to the locking securement of the plunger after stage 5.

While a number of exemplary aspects and embodiments have been discussed above, those of skill in the art will recognize certain modifications, permutations and additions and subcombinations thereof. It is therefore intended that the following appended claims and claims hereinafter introduced are interpreted to include all such modifications, permutations, additions and subcombinations that are within their true spirit and scope.

1. A safety syringe comprising:
an outer casing having a closed and an open end;
a transition chamber having a closed and an open end;
a spring assembly; and
a plunger;
the transition chamber being held for forced longitudinal movement within the outer casing and defining a needle concealment chamber formed between the closed ends of the outer casing and the transition chamber;
the plunger defining an enclosed needle receiving chamber and being held for sliding longitudinal movement within the transition chamber;
a liquid medicine chamber within the transition chamber, the transition chamber further defined between a proximal end of the plunger and the closed end of the transition chamber;
the spring assembly, being in a compressed configuration, including a needle within a needle containment chamber being supported between, the proximal closed end of the transition chamber and the closed end of the outer chamber, a distal end of the needle positioned within or immediately aligned with, but not through, a needle exist hole formed through a closed end of the outer casing;
movement of the plunger and the transition chamber together to make contact between the closed ends of the transition chamber and outer casing extending the needle from the needle exit hole;
further movement of the plunger within the transition chamber to make contact between a proximal closed end of the plunger and a proximal end of the spring assembly expels liquid medicine within the medicine chamber through and from the needle;
still further movement of the plunger within the transition chamber to make contact the closed ends of the transition chamber and the plunger retracts the needle into the needle exit hole by release of the spring assembly to an extended configuration and releases the spring assembly to eject freely into the needle receiving chamber.

2. A safety syringe comprising:
an outer casing having a closed and an open end;
a transition chamber having a closed and an open end;
a spring assembly; and
a plunger;
the transition chamber being held for forced longitudinal movement within the outer casing and defining a needle concealment chamber formed between the closed ends of the outer casing and the transition chamber;
the plunger defining an enclosed needle receiving chamber and being held for sliding longitudinal movement within the transition chamber;
a liquid medicine chamber within the transition chamber, the transition chamber further defined between a proximal end of the plunger and the closed end of the transition chamber;
the spring assembly, being initially in a compressed preloaded configuration, including a needle within a needle containment chamber being supported between, the proximal closed end of the transition chamber and the closed end of the outer chamber, a distal end of the needle positioned within or immediately aligned with, but not through, a needle exist hole formed through a closed end of the outer casing;
a first stage of movement being the positioning of the plunger and the transition chamber together into the outer casing into contact between the closed ends of the transition chamber and outer casing to extend the needle from the needle exit hole;
a second stage of movement of the plunger into the transition chamber making contact between a proximal closed end of the plunger and a proximal end of the spring assembly to expel liquid medicine within the medicine chamber through and from the needle;
subsequent movement of the plunger within the transition chamber so as to make contact the closed ends of the transition chamber and the plunger retracting the needle into the needle exit hole by release of the spring assembly to an extended configuration and releasing the spring assembly to allow the spring assembly to eject freely into the needle receiving chamber.

3. A single use pre-loaded disposable safety syringe comprising:
an outer casing having a closed and an open end;
a transition chamber having a closed and an open end;
a spring assembly; and
a plunger;
the transition chamber being held for forced longitudinal movement within the outer casing and defining a needle concealment chamber formed between the closed ends of the outer casing and the transition chamber;
the plunger defining an enclosed needle receiving chamber and being held for sliding longitudinal movement within the transition chamber;
a liquid medicine chamber within the transition chamber, the transition chamber further defined between a proximal end of the plunger and the closed end of the transition chamber;
a quantity of liquid medicine held within the liquid medicine chamber;
the spring assembly held in a compressed configuration including a needle extending within a needle containment chamber, the needle containment chamber defined by the proximal closed end of the transition chamber and the closed end of the outer chamber, a distal end of the needle positioned within or immediately aligned with, but not through, a needle exist hole formed through a closed end of the outer casing;
movement of the plunger carrying the transition chamber into the outer casing so as to make contact between the closed ends of the transition chamber and outer casing and extending the needle from the needle exit hole;
further movement of the plunger into the transition chamber so as to make contact between a proximal closed end of the plunger and a proximal end of the spring assembly expelling the liquid medicine within the medicine chamber through and from the needle;
still further movement of the plunger into the transition chamber so as to make contact the closed ends of the transition chamber and the plunger, releasing the spring assembly to an extended configuration retracting the needle back into the needle exit hole and releasing the extended spring assembly to eject freely into the needle receiving chamber.

4. A method of injecting liquid medicine into a patient comprising the steps of:
removing a safety cap from a proximal end of a safety syringe;
depressing a plunger of the syringe to a first stage of movement within an outer casing of the syringe to extend a needle into a patient from concealment within a needle concealment chamber defined between closed ends of the outer casing and a transition chamber of the syringe;
depressing the plunger to a second stage of movement into the outer casing to inject liquid medicine into the patient through the needle from a medicine chamber within the transition chamber;
depressing the plunger to a third stage of movement into the outer casing to dislodge a distal plug frictionally held within a mating seat formed into a proximal closed end of the plunger;
depressing the plunger to a fourth stage of movement into the outer casing to retract the needle back into a needle receiving chamber within the plunger;
depressing the plunger to a fifth stage of movement into the outer casing to release the needle into the needle receiving chamber and to secure the plunger from being withdrawn from the outer casing.
